(19) United States
(12) Patent Application Publication
(10) Pub. No.: US 2004/0102741 A1
(43) Pub. Date: May 27, 2004

(54) Container for dispensing unit doses of antibiotic ointment

(76) Inventor: Pamela Paulhus, Somers, NY (US)

Correspondence Address: MORGAN, LEWIS & BOCKIUS, LLP., 3300 HILLVIEW AVENUE, PALO ALTO, CA, 94304, US

(21) Appl. No.: 10/307,379

(22) Filed: Nov. 26, 2002

(51) Int. Cl. A61M 35/00

(52) U.S. Cl. 604/290

(57) ABSTRACT

A sturdy, convenient, and sanitary container for dispensing unit doses of antibiotic balm, wherein the unit doses are carried on separator strips, and stacked in a firm walled, puncture resistant container with reseal-able opening, and moved to the opening for dispensing.

### BACKGROUND OF THE INVENTION

Over-the-counter antibiotic ointment, or balm, has been available to the consumer in tubes made of metal or plastic, with a screw cap. Often, the tube is sealed when purchased. After the seal is broken, the cap may be used to reseal the tube, protecting the ingredients. Over time, the threads of the cap become filled with unused ointment, creating a mess, and making it difficult to reseal the tube.

In addition, the tube may become punctured, and ooze ointment. In either of these circumstances, the aseptic integrity of the ointment is called into question. In actuality, after just the first administration of ointment from the tube, the aseptic integrity of the remaining ointment may be affected by the means used to separate the ointment desired from the open end of the tube. All of these disadvantages are increased when the tube is not stored on the shelf of a medicine cabinet, but is stored in e.g., but not exclusive to, a diaper bag, purse, or pocket of clothing being worn by the individual.

### SUMMARY OF THE INVENTION

The present invention provides unit doses of antibiotic ointment in a container from which the ointment may be withdrawn, or dispensed, without contaminating the remaining ointment in the container. The container has a sturdy cylindrical wall, which resists puncture. In addition, the container is provided with a firmly fitted cap for the top dispensing opening, and a bottom closure, which seal the antibiotic in the container. Preferably, the cap is not a screw cap, but a cap with a firm friction fit, or expansion fit.

Each of the unit doses of the ointment in the container is stored on a separator strip. The individual doses, on their separator strips, are then stacked and placed within the container. Each of the separator strips may be provided with a tab. When grasped by its tab, the topmost dose, with its separator strip, may be removed from the remainder of the stack. If desired, the tab and the separator strip may be used as an applicator, for applying the ointment to a wound.

Within the container, the stack of separator strips and doses ride on a base which is provided with means for moving the base, and the unit doses in the lower portion of the stack, toward the opening for dispensing. In one preferred embodiment of the present invention, the bottom closure includes a rotatable disk with grooved surface, attached to a rotatable center pin. The center pin has a threaded outer surface which passes through an opening, with interior threads, in the base. By rotating the movable disk, the base moves (up) toward the opening, or (down) back toward the disk. The container may also be provided with a window, to indicate the level of antibiotic remaining in the container.

It is an object of the present invention to provide antiseptic antibiotic ointment in a dispenser, which overcomes the above difficulties. The ointment is provided in single doses, dispensed from a container with sides that cannot be pierced, and which can remain capped for extended periods of time with assured non-contamination of the antibiotic ointment if used as instructed.

These objects, as well as other objects which will become apparent from the discussion that follows, are achieved, in accordance with the present invention.

For a full understanding of the present invention, reference should now be made to the following detailed description of the preferred embodiments of the invention as illustrated in the accompanying drawings.

### BRIEF DESCRIPTION OF THE DRAWINGS

FIG. 1 is an exploded side view of the preferred embodiment of the container of the present invention for dispensing unit doses of e.g. antibiotic balm.

FIG. 2 is a side view, in partial cross section, of the bottom closure of the preferred embodiment shown in FIG. 1.

FIG. 3 is a partial cross sectional side view of an alternative embodiment of the container of the present invention.

### DESCRIPTION OF THE PREFERRED EMBODIMENTS

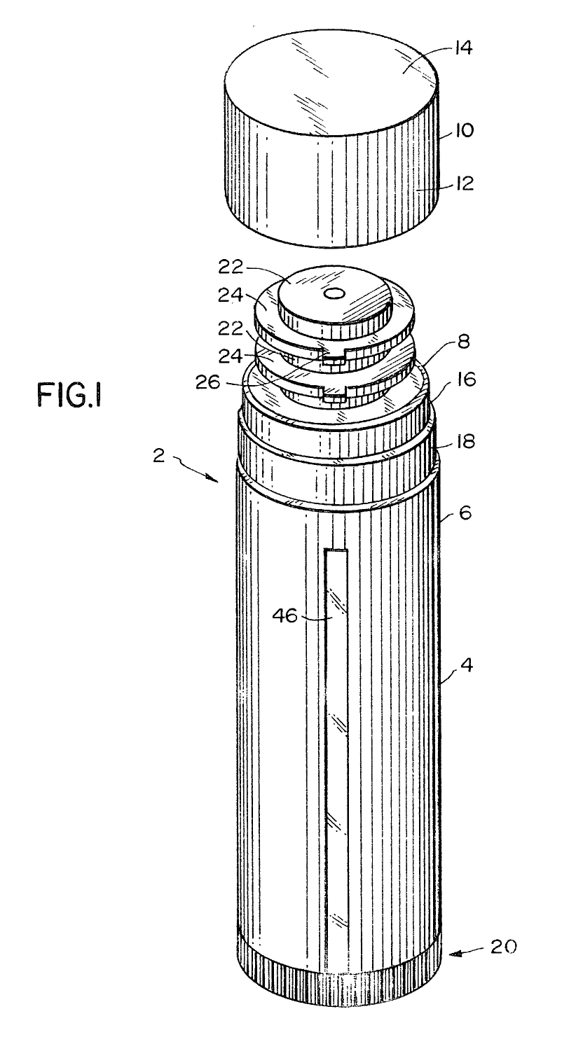

The preferred embodiments of the present invention will now be described with reference to FIGS. 1-3 of the drawings. Identical elements in the various figures are designated with the same reference numerals.

As shown in FIG. 1, the container of this preferred embodiment of the present invention is shown generally at 2. The container comprises a cylindrical side wall, 4. Though other configurations may be useful in forming the container of the present invention, it is the cylindrical container that most resists puncture. At the top, 6, of the side wall, is an opening, 8, through which the antibiotic balm is dispensed.

The container also comprises a cap, 10, with a cylindrical wall, 12, and top, 14, which covers the dispensing opening, and the top of the side wall of the container. At the top of the side wall, the container has means for making a firm closure of the cap over the opening. In the embodiment shown, these means comprises a bevel, 14, creating a reduced diameter in the top of the side wall, just slightly smaller than the interior diameter of cap, and a secondary bevel, of diameter intermediate that of the first bevel, and the container. Placing the cap over the beveled surface, 14, lines it up for making a firm friction fit as the side if the cap is pushed over secondary bevel, 18. Other means for creating a firm friction fit include, a roughened surface on the inside of the cap, and/or the beveled surfaces, and molded ridges on the inside of the cap, or on the top of the side wall.

As shown in FIG. 1, the unit doses, 22, each on a separator strip, 24, are stacked one atop the other to create a column of antibiotic balm for storage in, and dispensing from the container of the present invention. It is preferred that the separator strips, 24 include a tab, as shown at 26. By grasping the tab the user may easily pull a unit dose from the top of a stack, also assuring non-contamination of the antibiotic ointment by avoiding contact of the ointment by the grasping tab provided, a key feature of this design.

The antibiotic balm may comprise one of many compositions, varying in consistency from a very thick lotion to a waxy ointment. It may be necessary to provide a release coating to the upper and/or lower surface of the separator strips. A release coating will facilitate the removal of the topmost dose with separator sheet from the next underlying dose. In addition a release coating on the upper surface of the separator sheet will facilitate transfer of the balm from the separator sheet to a wound, especially a moist wound. The separator strip and tab may also provide a convenient and sanitary applicator as aforementioned.

As also shown in FIG. 1 the container has a transparent window 46, through which the contents of the container may be seen. The window may be made of translucent, transparent, or preferably transparent material, to permit viewing of the contents. The window is oriented along the length of the container, so as to reveal the level, or amount, of the remaining balm.

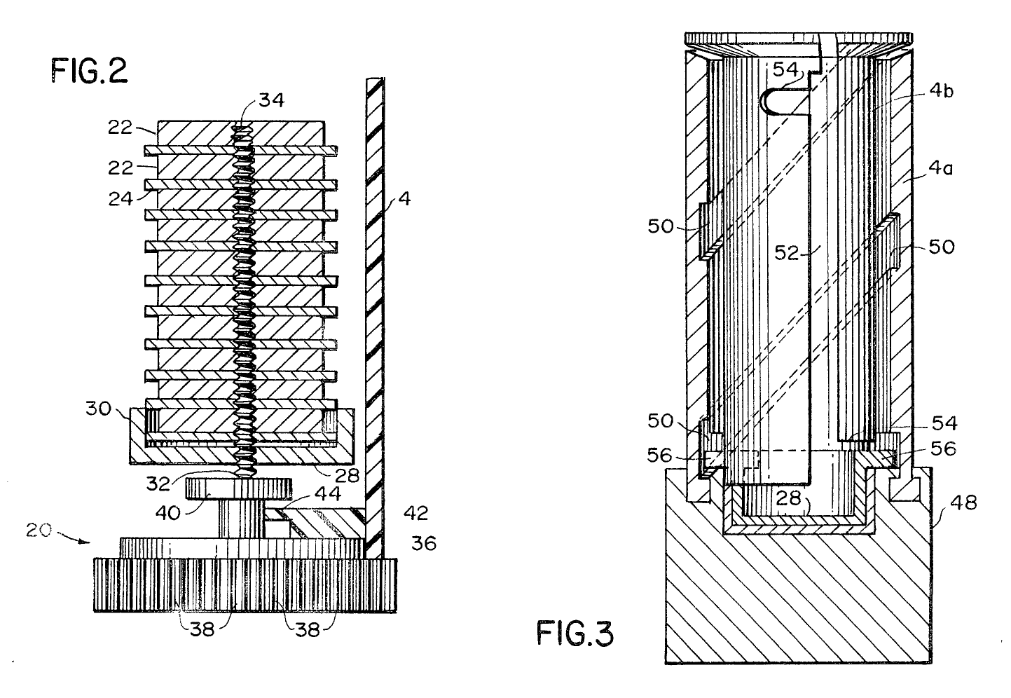

The bottom closure of the container shown in FIGS. 1 and 2, is shown generally at 20. In the embodiment shown the bottom closure comprises a number of elements which provide the means for moving the unit doses to the dispensing opening; and secure the side walls to the bottom of the container. As shown, a stack or column of antibiotic balm is disposed on the base, which is provided with lip, 30. The means for raising the base so as to continue dispensing sequential doses of the antibiotic balm until the supply in the container is exhausted comprises a disk, 36, with a centrally located perpendicular pin, 34, having a threaded outer surface. The pin passes through an opening, 32, in the base, bearing interior threads complementary with the exterior threads of the pin. Thus, by rotating the disk, 36, the base, 28, goes up or down depending on the direction of rotation. In the container shown in FIG. 1, provision of the center pin, 34, results in unit doses, 22, which are donut-shaped. In applying a unit dose to the skin at an e.g. wound site, as pressure is applied to the dose, it flattens and expands. In applying a donut-shaped dose, a portion of the dose is permitted to move toward the center, reducing the outward expansion of the dose, permitting a more accurate, and neater, application.

As shown in FIG. 2, the bottom closure preferably includes a pedestal, 40, located between the disk and the pin; and the side wall, 4, is provided with a stabilizing ledge, 42, with flexible tip, 44. The bottom of the stabilizing ledge rests against the top surface of the disk. It is important that these surfaces strip easily against each other. The flexible tip permits the pedestal to be pushed past the ledge, thus attaching the bottom (the disk) to the side wall of the container. The attachment and stabilization of these pieces assist in the smooth operation of the means for moving the base, and the unit doses disposed thereon, toward the dispensing opening. As shown in both FIGS. 1 and 2, the exterior or the perimeter of the disk may be provided with grooves, 38, to facilitate the rotation of disk 36.

In its preferred construction, the container is manufactured from plastic. High tensile plastic is used in the side walls, for strength and puncture resistance, and in the grooved disk, for durability of an external, moveable part. The cap is formed of soft plastic, to increase the grasp of the friction fit of the cap to the container. The threaded center pin and base are formed of softer plastic, to facilitate the manufacture of their threaded surfaces.

FIG. 3 illustrates an alternative embodiment of the container of the present invention. In this embodiment, the side walls comprise two layers; and outer layer, 4a, which turns against the inner layer, 4b. Inner layer 4b is fixed to the bottom, 48, of the container. The inner surface of the outer layer, 4a, is provided with two helical grooves, 50. The inner layer, 4b, has a vertical track, or cut-out, 52, with upper and lower stops, 54. The base, 28, is provided with posts, 56, which extend through the track and stops of 4b, into the grooves of 4a. By turning the outer layer 4a and the bottom 48 in opposite directions, the base is carried up and down between the stops. Reversing the direction of turning, reverses the direction of travel of the base. A stack of unit doses of antibiotic ointment or balm, with separator sides, may be disposed on the base for easy, sanitary, dispensing of the antibiotic. In this embodiment, the cap, not shown, must extend over the top opening, and down to the bottom, 48. A window in the cap may be aligned with the vertical track to view the level of antibiotic balm remaining in the container.

In still another embodiment, the ointment or balm may comprise, not a stack of unit doses, but a solid cylinder or column of antibiotic balm.

While the invention has been described with reference to an antibiotic balm, it is to be noted that either or both bacteriostatic or bacteriocidal ingredients may be used in the ointment or balm to be dispensed from the container. Other ingredients may also be added to the balm, to impart further, advantageous characteristics to the balm, which do not contradict the antibiotic properties of the balm.

There has thus been shown and described a novel container for dispensing single doses of antibiotic balm, which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

I claim:

1. A sturdy, convenient and sanitary container for dispensing antibiotic balm, comprising:
a cylindrical side wall having an opening at the top of the side wall, and a bottom closure at the bottom of the side wall;
a base on which a column of antibiotic balm may be disposed;
means for moving said base, and the column of antibiotic balm, to the opening;
a removable and replaceable cap, together with means for making a firm closure of the cap over the opening.

2. A container as in claim 1, wherein the column of antibiotic balm comprises a stack of unit doses of balm, between separator strips.

3. A container as in claim 1, wherein the separator strips have tabs.

4. A container as in claim 1, wherein the separator strips have a release coating.

5. A container as in claim 1, wherein the container further comprises a window for viewing the remaining balm.

6. A container as in claim 1, wherein said bottom closure comprises a pedestal attached to a disk, and a side wall stabilizing ledge with flexible lip for receiving the pedestal.

7. A container as in claim 6, wherein the means for moving the base comprises an externally threaded center, perpendicular pin attached to said pedestal, and extending through a centered, internally threaded opening in the base.

8. A container as in claim 7, wherein the unit doses have a centrally located opening.

9. A container as in claim 6, wherein the perimeter of the moveable disk is grooved.

10. A container as in claim 1, wherein the means for moving said base comprises a grooved surface disk attached to a threaded center pin, passing through a threaded opening in the base.

11. A container as in claim 1, wherein the means for making a firm closure of the cap over the opening, comprises a friction fit bevel.

12. A container as in claim 1, wherein the side wall comprises an inner layer, fixed to said bottom closure, and having a cut out vertical track and stops; and an outer layer with helical grooves on its inner surface, and wherein said base further comprises vertical posts which extend through the inner layer and into the grooves of the outer layer, whereby the base may be moved up and down by moving the outer layer in a direction opposite the inner layer and bottom.
